(19) United States
(12) Patent Application Publication
(10) Pub. No.: US 2008/0243018 A1
(43) Pub. Date: Oct. 2, 2008

(54) SYSTEM AND METHOD TO TRACK A RESPIRATORY CYCLE OF A SUBJECT

(75) Inventors: Joel F. Zuhars, Haverhill, MA (US); Vernon T. Jensen, Draper, UT (US); Thomas C. Kienzle, Lake Forest, IL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/694,060

(22) Filed: Mar. 30, 2007

(51) Int. Cl. A61B 5/08

(52) U.S. Cl. 600/534

(57) ABSTRACT

A system operable to track a respiratory cycle of a subject is provided. The system includes at least a first sensor positioned on the subject, and at least a second sensor located at a reference relative to a change in position of the first sensor associated with respiration of the subject. The system also includes a respiratory cycle measurement device coupled to receive the position data of the first sensor relative to the reference. The respiratory cycle measurement device is configured to translate the position data of the first sensor relative to time into a respiratory signal representative of a respiratory cycle of the subject.

### BACKGROUND OF THE INVENTION

The subject matter described herein generally relates to a system and method to monitor physiological activities of a subject, and more particularly to a system and method to monitor or track a respiratory cycle of a subject.

Many types of medical procedures involve devices where a change in position or orientation of an imaged subject is undesired. For example, a radiation therapy involves medical procedures where exposure of a non-cancerous tissue to high doses of radiation is undesired. In another example it is desired in radiation imaging, to direct radiation to only to a portion of the body to be imaged. Similarly, three-dimensional imaging applications such as computed topography (CT), PET, and MRI scans desire limiting direction of radiation to specific regions of interest of the imaged subject to be imaged. Other examples of medical procedures such as surgical procedures employing surgical navigation systems, desire accurate position and orientation information for navigating a surgical instrument relative to selected portions of the imaged subject.

A general limitation in the clinical planning and delivery of medical procedures such as those described above is the normal physiological movement associated with a living imaged subject. Normal physiological movement, such as respiration or heart movement, can cause a positional movement of the region of interest undergoing the medical procedure. Specifically in regard to radiation therapy applications, movement of a targeted region of interest may result in the radiation beam not being sufficiently sized or shaped to fully cover the targeted area. In regard to imaging applications, normal physiological movement may create blurred images or image artifacts. In surgical procedures, the normal physiological motion of the imaged subject can create undesired positional inaccuracies in navigation of the surgical instruments.

Thus, in general, motion associated with the physiological activity of the medical subject may influence the accuracy and efficacy of medical procedures (e.g., numerous types of surgical navigation, radiation therapy, and imaging).

Respiratory activity is a significant contributory factor in causing physiological movement of the imaged subject during many medical procedures. Several techniques have been used in diagnostic imaging to reduce motion associated with the respiratory activity. Breath holding has been used with success for many image acquisition applications and position critical surgical interventions, but this technique is not practical for radiation therapy as the radiation beam application time is typically too long for most imaged subjects to hold their respiratory activity.

Hence there is a need for a simple, accurate and low cost system to track respiration of an imaged subject. There also exists a need for a method to predict a respiration cycle of the medical subject.

### BRIEF DESCRIPTION OF THE INVENTION

The above-mentioned needs are addressed and can be understood by reading and understanding the subject matter described herein. Various other features, objects, and advantages of the subject matter described herein will be made apparent to those skilled in the art from the accompanying drawings and detailed description.

In one embodiment, a system to track a respiratory cycle of a subject is provided. The system includes at least a first sensor positioned on the subject, and at least a second sensor located at a reference relative to a change in position of the first sensor associated with respiration of the subject. The system also includes a respiratory cycle measurement device coupled to receive the position data of the first sensor relative to the reference. The respiratory cycle measurement device is configured to translate the position data of the first sensor relative to time into a respiratory signal representative of a respiratory cycle of the subject.

In another embodiment, a system to acquire an image data of an imaged subject is provided. The system includes an imaging system operable to acquire the image data of the imaged subject in communication with a respiratory cycle measurement device. The respiratory cycle measurement device is coupled to receive a position data of a first sensor at the imaged subject in relation to a second sensor at a reference. The respiratory cycle measurement device translates the position data of the first sensor relative to time into a respiratory signal representative of a respiratory cycle of the imaged subject.

In yet another embodiment, a system to gate delivery of radiation from a radiation source to a subject is provided. The system includes a respiratory cycle measurement device in communication to receive a position data of a first sensor at the imaged subject in relation to a second sensor at a reference. The respiratory cycle measurement device is configured to convert the position data over time into a respiratory signal, and to translate the respiratory signal into a gate signal. The system also includes a control unit in communication to receive the gate signal from the respiratory cycle measurement device. The gate signal causes the control unit to gate delivery of radiation from the radiation source to the subject relative to a respiration cycle of the subject.

Another embodiment of a system operable to navigate instruments relative to an image data of an imaged subject is provided. The system includes a respiratory cycle measurement device coupled to receive a position data of a first sensor attached to a patient in relation to a reference. The respiratory cycle measurement device translates the position data relative to time into a respiratory signal representative of a respiratory cycle of the imaged subject. The system also includes a controller operable to continuously reposition an image data relative to limits of a displayed image based on the position of the first sensor relative to the reference.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 is a schematic diagram of an embodiment of a tracking system.

FIG. 2 is a flow diagram of an embodiment of a method of monitoring respiratory cycle.

FIG. 3 is a graphical representation of a respiratory cycle of an imaged subject.

FIG. 4 is a block diagram of an embodiment of a system to gate communication of image data.

FIG. 5 is a block diagram of an embodiment of a processor assembly.

FIG. 6 is a schematic diagram of an embodiment of a system to measure or track a respiration cycle of a patient.

FIG. 7 is a schematic diagram of another embodiment of a system to measure or track a respiration cycle of a patient.

FIG. 8 is a flow diagram of another embodiment of a method of repositioning image data adjusted according to a respiration cycle of a patient.

FIG. 9 is a schematic diagram illustrative of an embodiment of repositioning image data in a region of interest of an acquired image.

FIG. 10 is a block diagram of an embodiment of a system to gate transmission of radiation.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

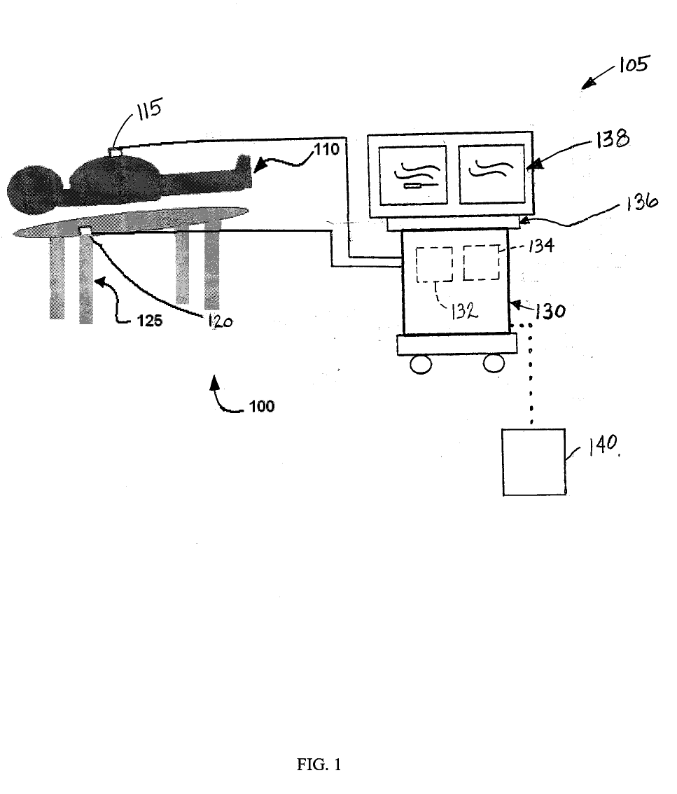

FIG. 1 is a schematic diagram of an embodiment of a system 100 operable to measure or monitor or track a respiration cycle. A technical effect of the system 100 is to gate acquisition of images and/or radiation therapy. The system 100 comprises a tracking system 105 configured to monitor the respiratory cycle of a medical subject or patient 110. The patient or subject 110 refers to a person or an animal receiving a medical procedure (e.g., imaging, radiation therapy, surgery, etc.). Yet, it should be understood that the system 100 can also be applied in other environments (e.g., industrial, etc.) to a variety of subjects 110 and is not limited solely to the medical field.

The tracking system 105 is generally operable to characterize a variable position associated with the respiratory mechanics of the patient 110. An embodiment of the tracking system 105 comprises at least a first sensor 115 positioned at the patient 110 (e.g., chest), and at least a second sensor 120 located at a reference relative to movement of the first sensor 115. The number of first sensors 115 or second sensors 120 can vary. One embodiment of the reference includes a table or patient positioning assembly 125 supporting the patient 110, or an imaging system operable to acquire an image data of the patient 110. Yet, it should be understood that the reference is not limited to the above-mentioned examples and can vary (e.g., the floor or a wall of the room selected to provide the medical procedure, etc.).

The first sensor 115 is positioned at the patient 110 such that the first sensor 115 moves in correlation with the respiratory cycle (e.g., inhalation and exhalation of lungs) of the patient 110. For example, the first sensor 115 can be positioned on the chest of the patient 110 so as to track the respiratory cycle of the patient 110. The second sensor 120 can be configured to detect, measure or sense a variable position such as an actual position and/or changes in position of the first sensor 115 and to translate the detected or sensed variable position into a position data relative to the first sensor 115. Either the first sensor 115 or the second sensor 120 can be configured to measure and transmit the sensed position data relative to the other sensor 115 or 120.

The tracking system 105 further comprises a respiratory cycle measurement device 130 coupled in communication with the first sensor 115 and/or second sensor 120. The type of communication (e.g., harness, wireless, internet, etc.) can vary. The respiratory cycle measurement device 130 is generally operable to generate a respiratory signal indicative of the respiratory cycle of the patient 110 based upon the position data received from the at least one second sensor 120. The respiratory cycle measurement device 130 can be independent of or can be integrated with the second sensor 120. An embodiment of the respiratory cycle measurement device 130 generally includes a processor 132 and a memory 134 to store programmable instructions for execution by the processor 132.

In an alternative embodiment, either the first sensor 115 or the second sensor 120 may not be in direct communication with the respiratory cycle measurement device 130. Accordingly, the first or second sensor 115 or 120 may send a sensor data to the processor 132, the sensor data corresponding to position of the first sensor 115 and/or change in position of the first sensor 115 relative to the second sensor 120. Further, the processor 132 may be configured to compute the position data based on the sensor data and consequently send the position data to the respiratory cycle measurement device 130.

Additionally, the tracking system 105 may include an interface 136 (e.g., mouse device, keyboard or keypad, touch-monitor, etc.) and a display 138 (e.g., monitor, LEDs, audible speaker, etc.) coupled to the respiratory cycle measurement device 130. The display 138 may be configured to illustrate the respiratory signal associated with the patient 110 for viewing. The tracking system 105 can also be connected in communication (e.g., wired, wireless, internet, etc.) with a remote workstation or receiver 140.

An embodiment of the tracking system 105 can be electromagnetic based or optical-based to generate data indicative of respiratory activity. Accordingly, each of the first sensor 115 or the second sensor 120 may include an optical sensor, an electro magnetic sensor, or any other sensing device or combination thereof operable to sense a changeable or variable position relative to one another and to generate an electrical output, such as a linear electrical output (LEO) or a digital electrical output (DEO), representative of the changeable or variable position during respiration. The electrical output of the first sensor 115 and/or the second sensor 120 can be expressed as, voltage potential, current, or other measurable electrical form. The tracking system 105 may receive power from an AC source, and/or from rechargeable or non-rechargeable batteries.

In another embodiment, the tracking system 105 can comprise a plurality of first sensors 115 or a plurality of second sensors 120 connected in communication (e.g., harness, wireless, internet, etc.) with the respiratory cycle measurement device 130. In a scenario comprising multiple first sensors 115 and multiple second sensors 120, each of the first sensors 115 can be tracked from each of the second sensors 120 in the tracking system 105. Although FIG. 1 shows the tracking system 105 having a first sensor 115 and a second sensor 120, it is understood that the number of first sensors 115 and second sensors 120 may vary. Also, the first sensor 115 and/or second sensor 120 can be a wireless sensor and may draw power from the tracking system 105 or may have a separate power source, such as a battery or photocell, for example. In yet another embodiment of the tracking system 105, the sensor 115 can be located at a ventilator system or ventilation detection device. The sensor 115 can be operable to generate a signal representative of a respiratory cycle correlated to a detected change in position or displacement of a ventilator component of the ventilator system or ventilation detection device with respiration of the patient 110.

Having provided the above description of general construction of the system 100, the following is the description of a method 200 of tracking or monitoring the respiratory cycle of the patient 110.

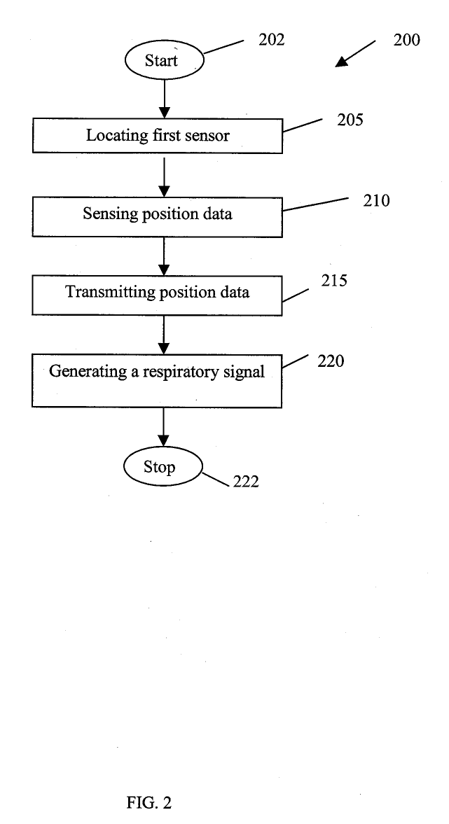

FIG. 2 illustrates a flow diagram depicting one embodiment of the method 200 of tracking a respiratory cycle of the patient 110. Step 202 is a start of the method 200. Step 205 includes positioning the at least one first sensor 115 on the patient 110. Step 210 includes sensing via the at least one second sensor 120 position data associated with the at least one first sensor 115. Step 215 includes transmitting sensed position data of the first sensor 115 via the at least one second sensor 120. Step 220 includes generating a respiratory signal indicative of the respiratory cycle of the patient 110 based upon received position data associated with the at least one first sensor 115. Step 222 is the end of the method 200.

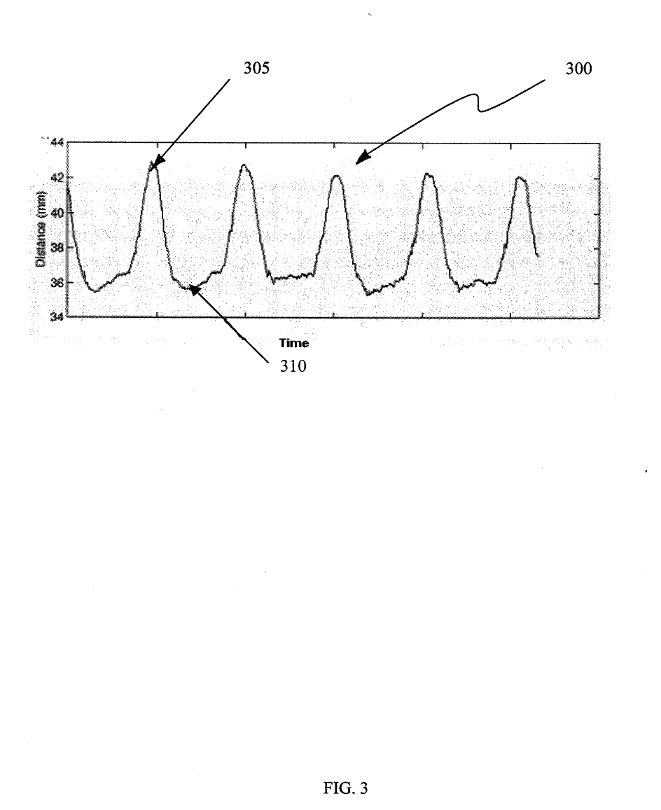

FIG. 3 displays a time-varying plot of the respiratory signal 300, generated at, the tracking system 105. The respiratory signal 300 can be used as a real-time indicator of the motion representative of a respiratory cycle of the patient 110. The respiratory signal 300 graphically represented in FIG. 3 depicts the changes in position of the first sensor 115, as measured by the second sensor 120, in relation to time, as measured in seconds.

The respiratory signal 300 generated by the system 100 can aid in collecting statistics or displaying information about the respiratory activity of the patient 110. Each respiratory signal 300 (as illustrated in FIG. 3) representative of the respiratory cycle of the patient 110 comprises a stream of digital data samples that collectively form a signal wave pattern representative of the respiratory cycle. The stream of data samples can be taken during a given time period. For example, approximately 200-210 data samples are measured for each approximately 7-second time interval.

A pattern-matching analysis can be performed against the measured data samples. In an embodiment, the most recent set of data samples for the respiratory signal 300 is correlated against an immediately preceding set of data samples to determine the period and repetitiveness of the respiratory signal 300. Thus, the pattern matching analysis provides a tool for measuring the periodicity of the respiration signal 300, thus allowing detection of deviation from a normal respiratory motion. The pattern matching analysis can be used during radiation therapy, imaging, and interventional procedures that are facilitated or require monitoring of the respiratory motion of the patient 110. The pattern matching analysis can also be used to predict the respiration signal 300, including a future time of exhalation and inhalation, of the patient 110.

As illustrated in FIG. 3, the respiratory signal 300 is generally sinusoidal in nature, with the least a motion or change in position occurring at a maximum or peak of inhalation 305 and a minimum or peak of exhalation 310. At the peak points 305 and 310 in the respiratory signal 300, the motion of the patient 110 is at a minimum. The optimal time either to acquire image data or to activate the radiation beam of a radiation therapy device is at the peak points 305 and/or 310 with the least motion or movement of patient 110. Therefore, by sensing the respiratory peaks 305 and 310 of least motion, and timing image data acquisition and instrument navigation to occur at the peak points 305 and 310, inaccuracies due to respiratory motion can be decreased. Additionally, acquiring the image data and patient position data at both peaks 305 and 310 facilitates the possibility to interpolate image data and patient position data to provide accurate navigation during the respiratory cycle. An embodiment of the tracking system 105 can be configured to receive an instruction (via an input device like a mouse, keyboard, or touch-screen, etc.) of a selection of a moment or location of the respiratory signal 300 to be designated as full exhalation or inhalation of the patient 110. The tracking system 105 can also be configured to receive an instruction of a selection of a moment or location (e.g., one or both of the peaks 305 and 310) in the respiratory signal 300 to trigger acquisition of the image data or transmission of radiation in radiation therapy.

In another embodiment, the respiratory signal 300 generated by the system 100 can be employed in a respiration responsive gating system. The respiration responsive gating system includes systems for controlling radiation in radiation therapy/imaging systems. In regard to radiation therapy, the respiratory responsive system 100 synchronizes application of radiation with the respiratory motion of the patient 110. In regard to image data acquisition, the system 100 synchronizes acquisition of image data with the respiratory motion of the patient 110.

One aspect of gating is to determine boundaries of gating intervals (e.g., duration of ON state) for applying radiation or acquiring image data. For gating purposes, a threshold can be defined over the amplitude range of the respiratory signal 300 to determine the boundaries of the gating intervals. For example, one boundary of gating interval can include a predetermined threshold of motion or movement of the patient 110. Unacceptable levels of movement outside the predetermined threshold can result from the respiratory cycle or from sudden movement or coughing by the patient 110. The motion of the first sensor 115 can be accepted as a representation of the motion of an internal anatomy of the patient 110.

In imaging applications, an example of a boundary of gating interval can include a predetermined respiratory motion that is predicted to increase the likelihood of image errors. Alternatively, a boundary of gating interval can include a predetermined respiratory motion that is predicted to correspond to fewer errors in image data acquisition.

In therapeutic applications, the gating intervals correspond to the portion of the respiratory cycle in which motion of a clinical target volume is minimized. The radiation is applied to the patient 110 when the respiratory signal 300 is within the boundaries of the gating intervals. Thus, the radiation beam pattern can be shaped with the minimum possible margin to account for the respiratory motion of the patient 110.

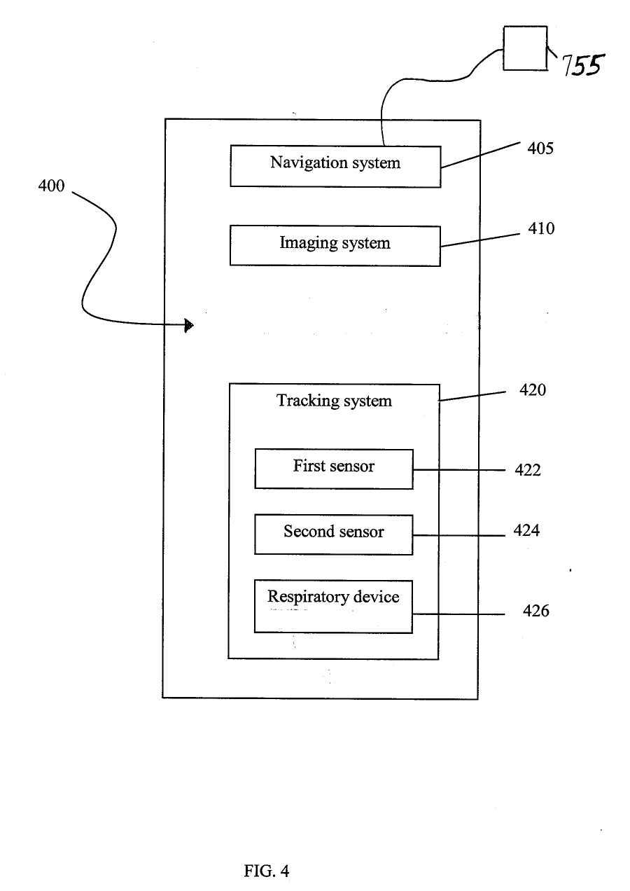

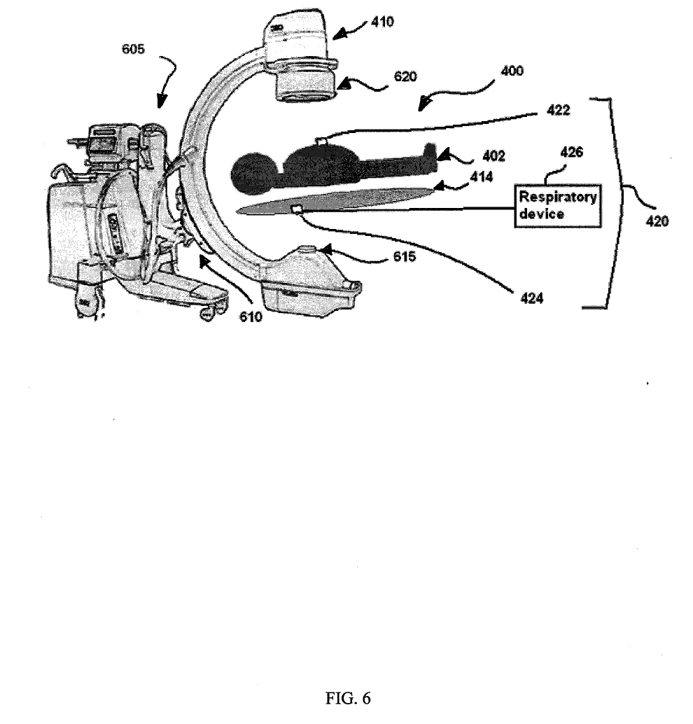

FIG. 4 represents a block diagram of an embodiment of a system 400 to gate communication of image data of a subject 402 (FIG. 6). The system 400 comprises a navigation system 405, an imaging system 410 operable to acquire the image data of the subject 402, and a tracking system 420 having at least one sensor 422 (may further include a second sensor 424 as a reference although not required) and a respiratory cycle measurement device 426, similar to the tracking system 105 having sensors 115 and 120 and respiratory cycle measurement device 130 as described above.

The navigation system 405, the imaging system 410, and the tracking system 420 are connected to be in communication with one another as part of a network. An example of the network includes a Local Area Network (LAN), such as an Ethernet, installed in a hospital or a medical facility. The network can be interconnected via a hard-wired connection (e.g., cable, bus, etc.) or a wireless connection (e.g., infrared, radio frequency, etc.) or combination thereof.

The navigation system 405 is generally operable to track the position and orientation of a surgical instrument (e.g., a surgical tool such as a bone drill, implant insertion device, a catheter, a guide wire, etc.), as well as to illustrate the position and orientation of the surgical instrument relative to an internal anatomy of the patient 110 as imaged using the imaging system 410. In one embodiment, the position and orientation of the surgical instrument can be tracked by the tracking system 420 as opposed to the imaging system 410, thereby alleviating the need to continually acquire the image data using the imaging system 410, and thereby reducing the amount of radiation exposure to the subject 402 and/or operating personnel.

The imaging system 410 can include a mobile or a fixed imaging system such as a computed tomography (CT) imaging system, a positron emission tomography (PET) imaging system, a magnetic resonance (MR) imaging system, an ultrasound imaging system, or an X-ray imaging system. One of ordinary skill in the art shall however appreciate that the imaging system 410 is not limited to the examples given above.

The imaging system 410 in communication with the navigation system 405 is configured to acquire image data associated with the subject 402. The imaging system 410 is further configured to transmit acquired image data along with a clock time to the navigation system 405.

In an alternate embodiment, the imaging system 410 is in analogue communication with the navigation system 405 and transmits a continuous video output of the acquired image data. The navigation system 405 receives the image data and computes the clock time thereby correlating the image data. For example, the imaging system 410 in combination with the navigation system 405 and the tracking system 420 acquires a series of images of the subject 402 at timed intervals between full inhalation and full exhalation in the respiratory cycle of the subject 402. The limits or moments defining the respiratory cycle can vary. The system 400 can be configured to correlate each of the series of acquired images with a moment or location in the detected respiratory cycle (e.g., a position or change in position versus time with respiration of the subject 402, a percentage of full exhalation or full inhalation of the subject 402, etc.). For example, a first image can be correlated to a first percentage (e.g., ninety percent of full inhalation or exhalation), and a second image can be correlated to a second percentage (e.g., fifty percent of full inhalation or exhalation) of full inhalation or exhalation of the subject 402. The system 400 can be configured to allow selection of one of the series of images correlated to the moment or position along the respiratory cycle, the image for superposition with a graphic representation of the location of the surgical tool, as tracked by the navigation system 405.

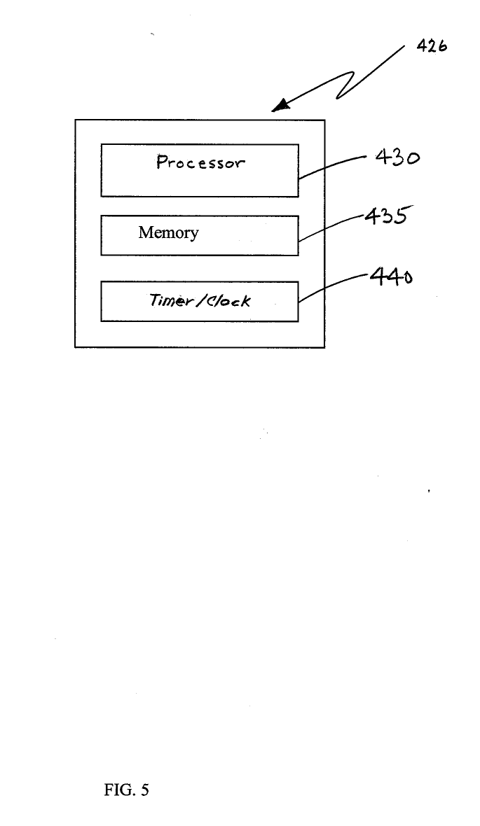

FIG. 5 is a block diagram of an exemplary embodiment of the respiration device 426. The respiration device 426 includes a processor 430 in communication with a memory 435 and a timer or clock unit 440. The memory 435 generally includes programmable instructions for execution by the processor 430 to process the position data associated with the respiratory signal 300 and thereby generate the gate signal. The memory 435 is also configured to store the respiratory signal 300. The timer or clock unit 440 is generally configured to generate a clock output signal. The processor 430 of the respiratory cycle measurement device 426 is generally operable to translate the respiratory data of the respiratory signal 300 into generate a gate signal. The gate signal can be an electrical output or a digital output comprising an ON state and an OFF state. The gate signal in combination with the clock output signal is generally operable to gate or regulate the radiation therapy or image data acquisition relative to the movement of the subject 402.

Upon receiving the respiratory signal 300 from the tracking system 420, the respiratory cycle measurement device 426 computes a change in position of the first sensor 422. The change in position of the first sensor 422 is compared with a threshold. The threshold corresponds to a suitable limit of movement or change in position of the first sensor 422 associated with an acceptable level of displacement induced by the respiration of the subject 402. The threshold can be selected and stored in the memory unit 510 of the processor assembly 415. The selection of the threshold determines the boundary of gating interval.

The respiratory cycle measurement device 426 is also configured to identify a predetermined gating event when a change in three-dimensional position of the first sensor 422 exceeds the threshold, and/or when a mathematical derivative (e.g., rate of change of) the respiratory signal 300 exceeds the threshold. The respiratory cycle measurement device 426 is configured to identify any predetermined subset period of a single respiratory cycle, including one or more individually identifiable positions within the single respiratory cycle, in either a continuous or sequenced manner.

Upon identifying the predetermined gating event, the respiratory cycle measurement device 426 is operable to generate an OFF state of the gate signal. Alternatively, the respiratory cycle measurement device 426 can be configured to generate an ON state of the gate signal when the change in position of the first sensor 422 is less than the threshold of the movement. The gate signal thus generated is further synchronized with the clock output signal generated by the timer unit 440.

The navigation system 405 is configured to correlate the gate signal with the image data so as to selectively gate the acquisition of the image data. For the purpose of gating imaging acquisition, the navigation system 405 is configured to accept the image data from the imaging system 410 upon detecting an ON state of the gate signal. Alternatively, the navigation system 405 can be configured to discard or prevent the transfer or use of the image data upon detecting an OFF state of the gate signal. The benefit of gating the image data results in an improvement in the navigation accuracy of the image data that are accepted by the navigation system 405.

Although the respiratory cycle measurement device 426 is shown integrated with the tracking system 420, the respiratory cycle measurement device 426 can be alternatively integrated with one or both of the navigation system 405 and the imaging system 410. Alternatively, the respiratory cycle measurement device 426 can be installed in an independent device. In a similar manner, although the processor 430, memory 435, and timer unit 440 are shown integrated with the respiratory cycle measurement device 426, it should be understood that one or more of the processor 430, memory 435, or timer unit 440 can integrated with one or more of the navigation system 405, imaging system 410, or tracking system 420 or as an independent system therefrom.

FIG. 6 illustrates a schematic diagram of the embodiment of the system 400 operable to acquire and/or to communicate acquired image data of the patient 402. The system 400 comprises the imaging system 410 having the tracking system 420 installed thereon. The imaging system 410 includes a conventional C-arm 412 positioned to direct a radiation beam at the subject 402 positioned on the patient positioning assembly 414, similar to the patient positioning assembly 125 described above. It should be understood that the system 400 may be used with other types of imaging systems (PET, MRI, ultrasound, mammogram, endoscope, etc.), therapeutic systems and in other applications.

The illustrated imaging system 410 comprises a main assembly 605, a mobile support assembly 610 coupled to the main assembly 605, at least one radiation source 615, and at least one radiation detector 620 configured to operate in conjunction with the radiation source 615. For mobile-type imaging systems 410, the support assembly 610 supports the radiation source 615 and/or the radiation detector 620. The support assembly 610 can include a structural C-shaped members or structural O-shaped members in support of the radiation source 615 and/or radiation detector 620. The main assembly 605 in combination with the support assembly 610 is operable to selectively move the radiation source 615 and the radiation detector 620 of the imaging system 410 to various positions so as to acquire image data (e.g., two-dimensional, three-dimensional) at different views of one or more regions of interest of the subject 402.

The tracking system 420 installed on the imaging system 410 comprises a first sensor 422 positioned on the subject 402 and the second sensor 424 positioned on the patient positioning assembly 414. Alternatively, the at least one second sensor 424 configured to sense position data associated with the at least one first sensor 422 can be coupled to the imaging system 410. Accordingly, the at least one second sensor 424 can be secured, attached, installed or mounted on the main assembly 605, the support assembly 610, the radiation source 615 or the radiation detector 620 of the imaging system 410.

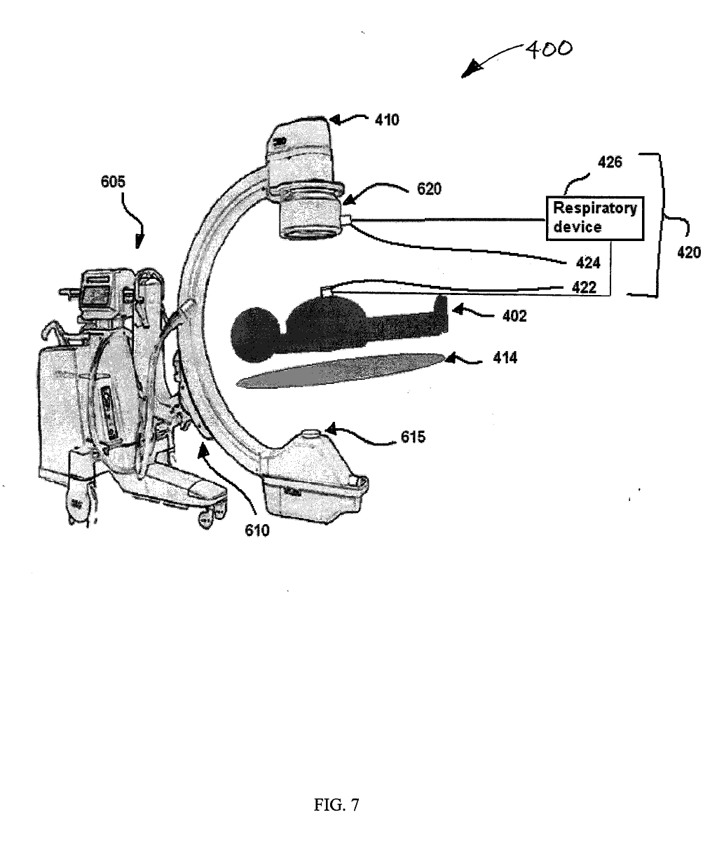

FIG. 7 is a schematic diagram of another arrangement of the embodiment of the system 400 operable to gate acquisition of image data. The system 400 comprises the at least one second sensor 424 positioned on the radiation detector 620 of the imaging system 410 near the area of interest and in communication with the first sensor 422.

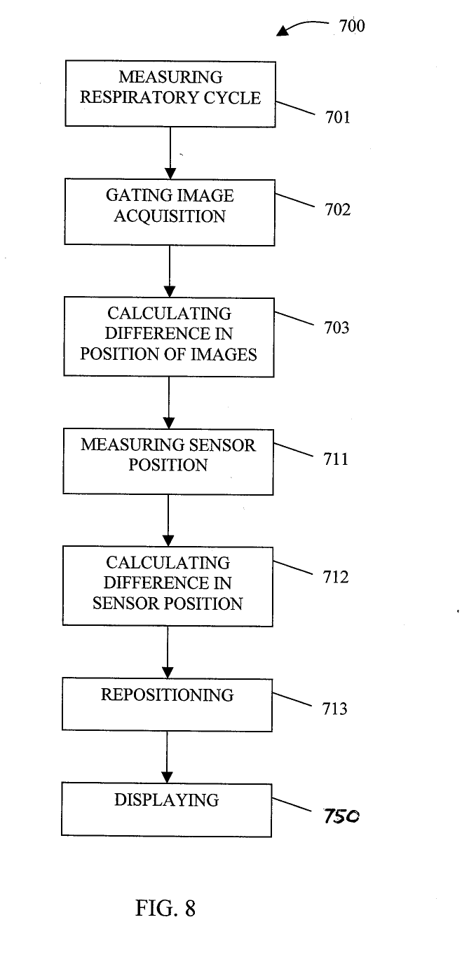

FIG. 8 includes a flow diagram illustrative of an embodiment of a method 700 to adjust a position of displayed image data based on the tracking of the respiratory cycle of the subject 402. Assume the first sensor 422 is positioned at the patient 402 and connected in communication with the respiratory cycle measurement device 426, which is in communication with the navigation system 405 and the imaging system 410. Step 701 includes detecting or measuring the respiratory cycle 300, via the respiratory cycle measurement device 426 in communication with sensor 421 measuring and recording the corresponding positions of the first sensor 422 associated with respiratory movement of the subject 402. Step 702 includes gating acquisition of images by the imaging device 410 via the navigation system 405 so as to acquire respective images of the position of the subject 402 correlating to an occurrence of the respiratory peaks 305 and 310 in the respiratory cycle 300 of the subject 402, and recording the position of the first sensor 422 corresponding to each acquired image. Step 703 includes calculating a difference in position between acquired images correlated to respective different points (e.g., respiratory peaks 305 and 310) or locations along the respiratory cycle 300 of the subject 402. For example, a 25 mm movement in the position of the first sensor 422 may correlate a 15 mm movement of the vertebrae (e.g., region of interest) of the subject 402 as illustrated and measured from acquired images. An example of the calculating the difference in position between images includes determining a difference in spatial relation of an outermost edge of captured image data in the two comparison images relative to a common reference. Another example includes calculating a difference in position or location of anatomical landmarks or references identified in the comparison images using known image processing techniques relative to a common reference.

Step 711 includes measuring or detecting the current position of the first sensor 422 on the subject 402. Step 712 includes calculating a difference or change in the current position of the first sensor 422 relative to a previous position of the first sensor 422 correlated to one of the previously acquired and stored images of the subject 402 described above. Step 713 includes repositioning the image data in a displayed image by an amount or spatial relation correlated to or dependent upon the difference or change in position of the first sensor 422 as calculated in step 712 above. An embodiment of repositioning generally includes moving the location of all or a portion of image data (e.g., region of interest in the current image) in the displayed image relative to a window defining an outer limits of the image data of an acquired image. The amount or spatial relation or repositioning is proportional to the difference in position of the first sensor 422 as calculated in step 712 above. Techniques to reposition the acquired image include interpolation and extrapolation, yet the type of technique can vary.

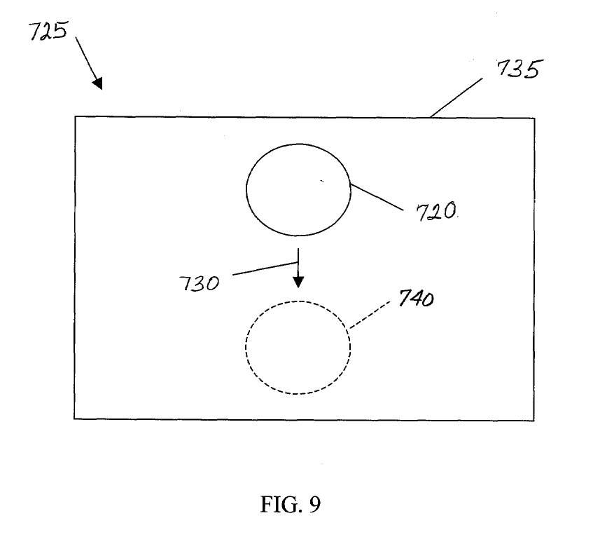

For example, assume an identified point of inspiration (e.g., peak point 305) in the respiration cycle 300 (FIG. 3) is correlated to a movement of 25 mm of the first sensor 422 (FIG. 4) relative to a reference. As shown to FIG. 9, accordingly, image data in an original region of interest 720 of a displayed image 725 is spatially repositioned or moved in a direction (illustrated by arrow and reference 730) in proportion to the measured movement of the first sensor 422 so as to achieve a real-time location of the respective image data in the region of interest 720 relative to the limits or window 735 of the displayed image 725. As shown in FIG. 9, the repositioned image data is illustrated in dashed line and by reference 740. The proportion or ratio of spatial repositioning can be predetermined according to stored data correlating movement of the sensor 422 relative to associated movement of the region of interest or anatomical landmark or reference.

Referring back to FIG. 8, step 750 includes displaying the representations of one or more tracked objects or instruments 755 (See FIG. 4) superimposed on the repositioned current image described above. Steps 711, 712, 713 and 750 are repeated during navigation of the objects or instruments 755 through the subject 402 in repositioning and displaying newly acquired images.

Alternatively, steps 702 and 703 may be repeated with the imaging system 410 aligned in more than one viewpoint so that multiple sets of images are collected, each set including images corresponding to one of the respiratory peaks 305 and 310 of the subject 402. Steps 712 and 713 may be repeated for each set of acquired images from each of the viewpoints of the imaging system 410, such that associated sets of multiple repositioned images calculated as described in step 713 are simultaneously displayed from each viewpoint of the imaging device 410. Similar to step 750, one or more representations of tracked instruments or objects can be superimposed on each set of the repositioned images.

In accordance with another alternative embodiment, one or more images may be acquired at arbitrary points of the respiratory cycle 300. During navigation of the object through the subject 402, a position of the sensor 422 is measured and techniques known in the art are employed to calculate an amount or spatial relation to reposition the acquired images. Similar to step 750 described above, representations of the tracked instruments or objects can be superimposed on the one or more repositioned images.

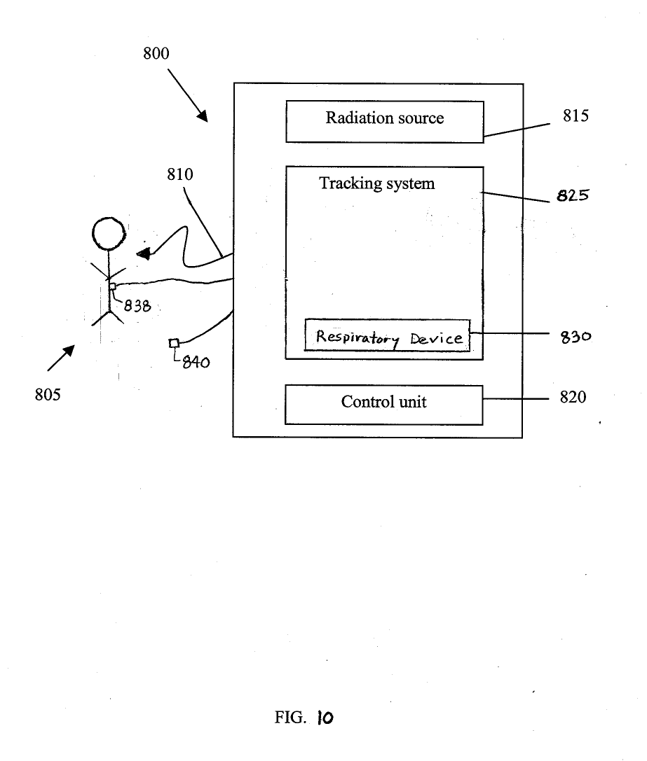

FIG. 10 illustrates an embodiment of a system 800 operable to gate exposure of the subject 805 to radiation 810, such as may be performed in a radiation therapy procedure. The system 800 is generally operable to synchronize exposure to or application of the radiation 810 from a radiation source 815 with the occurrence or non-occurrence of a predetermined gating event in a respiratory cycle of the subject 805. In addition to the radiation source 815, the system 800 includes a control unit 820, and a tracking system 825 having a respiratory cycle measurement device 830, connected in communication with one another and the radiation source 815. The radiation source 815, the control unit 820, and the tracking system 825 can be connected to be in communication with one another as part of the network installed in a hospital or a medical facility. The type of radiation 810 can include x-rays, electromagnetic radiation within visible or near visible frequency spectrum, an activating radio frequency (RF) field employed in MRI imaging, sonic radiation, or radiation in the form of a particle beam. The radiation source 815 is generally operable to generate and transmit or communicate the radiation 810. The radiation 810 may be directed to target sites or region of interest (ROI) that move with or are affected by the respiratory cycles. Such sites include, but are not limited to, the heart, the mediastinum, the lung, the breast, the kidney, the esophagus, the chest area, the liver, and the peripheral blood vessels. The radiation 810 may be also applied during a specific portion of the respiratory cycle to a site such as a tumor that does not move substantially but is nevertheless affected by the respiratory cycle.

Similar to the respiratory cycle measurement devices 130 and 426 described above, the respiratory cycle measurement device 830 of the tracking system 825 is operable to convert a respiratory signal 300 into the gate signal for communication to the control unit 820. In response to the gate signal, the control unit 820 regulates exposure or transmission of radiation 810 from the radiation source 815 relative to detected movement associated with respiration of the subject 805, similar to that described above with respect to the system 100 and system 400. An embodiment of the control unit 820 comprises an electrical switch operatively coupled to switch transmission of radiation 810 from the radiation source 815 in an ON and OFF manner. The switch can be operated to activate, enable activation of, or suspend the application of radiation 810 to the subject 805 based upon the gating signal. In one embodiment, upon detecting an OFF state of the gate signal generated by the tracking system 825, the control unit 820 causes deactivation of the radiation source 815. The radiation source 815 remains deactivated until the processor 825 generates an ON state in the gate signal which causes the control unit 820 to activate the radiation source 815 to generate and transmit radiation 810 directed towards the subject 805. The radiation source 815 remains activated until detecting an OFF state in the gate signal.

In an alternate embodiment, the control unit 820 enables activation of the radiation source 815, and the radiation source 815 can be activated and deactivated by a user such as a medical staff until detecting an OFF state in the gate signal.

Thus, a technical effect of the measurements carried out by the tracking system 825 includes generating the ON and OFF states of the gate signal, which in turn controls the activation and deactivation of the radiation source 815. The term “activates” is used in the broad sense to describe energizing or enabling the radiation source 815 to transmit radiation 810 directed to impinge upon the subject 805. Thus, the term is meant to encompass not only a situation where the radiation source 815 is normally dormant (e.g. an x-ray source requiring an electrical signal to trigger production of x-rays), but also a scenario where the radiation source 815 is one which continuously generates radiation 810 and “activation” of the radiation source 815 includes opening of a shutter or other occluding mechanism so as direct transmission of radiation 810 towards the subject 805.

In yet another embodiment, first and second sensors 838 and 840, (similar to the second sensor 120 and 424 described above) of the tracking system 825 can be configured to produce digital electrical output having ON and OFF states representative of the respiratory activity of the subject 805. The ON and OFF states represented in the digital electrical output can be directly communicated to control deactivation and activation of the radiation source 815.

The radiation source 815 can be integrated with a radiation therapy device in which application of radiation 810 to the subject 805 performs a therapeutic function, as opposed to diagnosis, in which radiation 810 is applied to perform a diagnostic or imaging function. Alternatively, the radiation source 815 can integrated with the imaging system 410 described above.

In another embodiment, the control unit 820 can be connected to control multiple radiation sources 815 coupled to multiple medical devices in accordance with the needs of the examination or treatment, such as radiation therapy apparatus, linear accelerator, CT, MRI, PET, SPECT, or ultrasound image acquisition apparatus, laser surgery apparatus, or lithotripsy apparatus. According to this embodiment, activation and deactivation of the radiation sources 815 of multiple medical devices can be executed for multiple medical procedures on the subject 805 and can be simultaneously controlled via the gate signal from the tracking system 825.

An embodiment of the power supply for the systems 100, 400, and 800 includes one or more batteries that can be removably mounted to facilitate replacement. The power supply, which can be rechargeable, can be adapted to supply electrical power to operate one or more sensors 115, 120, 422, 424, and/or the tracking systems 420 and 825, and/or the radiation source 815 and/or the control unit 820 and to power auxiliary elements such as the display 138 (See FIG. 1).

The above-description of systems 100, 400 and 800 provide a simple and low cost tracking of a respiratory cycle of a patient or subject 110, 402, and 805. Further, the systems 100, 400, and 800 and method 200 can be employed in various medical procedures, such as imaging, radiation therapy and surgery.

The respiratory signal 300 generated by the tracking system 105 can be used to control the acquisition of image data in imaging applications and to control the application of radiation in therapeutic applications. In 3-dimensional imaging applications such as CT, PET and MRI, the respiratory signal 300 is operable to retrospectively “gate” the reconstruction process. For this purpose, the acquisition of image data is synchronized to a common time base with the respiratory signal 300. Segments of the acquired image data that correspond to respiratory cycle intervals of interest are used to reconstruct the volumetric image data, thus minimizing the distortion and size changes caused by the motion of the subject 110, 402, and 805. Also, the above-described gated acquisition of image data enables use of a pre-surgical image data in place of inter-operation acquired image data, which can reduce overall radiation dosage.

The above-described system 800 to gate application of therapeutic or diagnostic radiation to a tissue volume of the subject 805 during a selected portion of the, respiratory cycle of the subject 805 diminishes inaccuracies in an assumed spatial position of the tissue volume arising from displacements induced by the respiratory motion of the subject 805.

The above-described systems 100, 400 and 800 and method 200 are also applicable to surgical applications that require real-time representations of time varying anatomy of the patient or subject 110, 402, and 805. The gating of the image data can enhance accuracy of the acquired navigation image data for illustration on the display 132 or the navigation system 405. The enhanced accuracy of the navigation image data can increase precision in locating the surgical instruments within the subject 110 and 805 resulting, in a less invasive surgical procedure and reducing risks associated with more invasive surgical procedures (e.g., open surgery).

Also, the above-described systems 100, 400 and 800 and method 200 can be implemented in connection with other applications, such as monitoring a physiological activity occurring in the subject 110 and 805 and gating the recording and displaying of data relative to the physiological activity.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system to track a respiratory cycle of a subject, the system comprising:
at least a first sensor positioned on the subject;
at least a second sensor located at a reference relative to a change in position of the first sensor associated with respiration of the subject; and
a respiratory cycle measurement device coupled to receive the position data of the first sensor relative to the reference, wherein the respiratory cycle measurement device is configured to translate the position data of the first sensor in relation to the reference relative to time into a respiratory signal representative of a respiratory cycle of the subject.

2. The system of claim 1, wherein the respiratory cycle measurement device includes a display device coupled in communication to receive and illustrate a display of the respiratory signal.

3. The system of claim 2, wherein the respiratory cycle measurement device includes a processor operable to generate a gate signal in based on a comparison of the position of the first sensor, wherein the gate signal is in an ON state when the position of the first sensor is within a first threshold of a peak position and a second threshold of a minimum position relative to a position of the second sensor, and wherein the gate signal is in an OFF state when the position of the first sensor is detected not within the first and second thresholds relative to the second sensor.

4. A system to acquire an image data of an imaged subject, the system comprising:
an imaging system operable to selectively acquire the image data of the imaged subject; and
a respiratory cycle measurement device coupled to receive a position data of a first sensor at the imaged subject in relation to a second sensor at a reference, wherein the respiratory cycle measurement device translates the position data relative to time into a respiratory signal representative of a respiratory cycle of the imaged subject.

5. The system of claim 4, wherein the reference comprises a positioning assembly configured to support the imaged subject or the imaging system, and wherein the tracking system includes at least a first sensor positioned at the imaged subject and at least a second sensor located at a reference relative to a position of the imaged subject, wherein the second sensor is configured to detect position data of the first sensor relative the second sensor.

6. The system of claim 4, wherein the respiratory cycle measurement device includes a processor operable to generate a gate signal correlated to the respiratory signal.

7. The system of claim 6, wherein the processor synchronizes the gate signal with a clock output signal.

8. The system of claim 7, wherein the processor compares the position data of the first sensor relative to a threshold, and wherein the gate signal is in an ON state when the position data of the first sensor is within the threshold, and wherein the gate signal is in an OFF state when the position data of the first sensor is detected not within the threshold.

9. The system of claim 7, wherein the processor calculates a change in position of the first sensor relative to the second sensor relative to the clock output signal, and wherein the gate signal is in the ON state when the change in position of the first sensor is within the threshold, and wherein the gate signal is in the OFF state when the change in position of the first sensor is detected not within the threshold.

10. The system of claim 6, further comprising a system operable to correlate the gate signal from the respiratory cycle measurement device with the image data received from the imaging system.

11. The system of claim 10, wherein the system is configured to accept communication of the image data from the imaging system in response to detecting an ON state of the gate signal.

12. The system of claim 10, wherein the system is configured to reject communication of the image data from the imaging system in response to detecting an OFF state of the gate signal.

13. The system of claim 10, wherein the system is configured to cause acquisition of the image data from the imaging system in response to detecting an ON state of the gate signal, and wherein the system is configured to stop acquisition of the image data from the imaging system in response to detecting an OFF state of the gate signal.

14. A system to gate delivery of radiation from a radiation source to a subject, the system comprising:
a respiratory cycle measurement device in communication to receive a position data of a first sensor at the imaged subject in relation to a second sensor at a reference, wherein the respiratory cycle measurement device is configured to convert the position data over time into a respiratory signal, and wherein the respiratory cycle measurement device is configured to translate the respiratory signal into a gate signal; and
a control unit in communication to receive the gate signal from the respiratory cycle measurement device, wherein the gate signal causes the control unit to gate delivery of radiation from the radiation source to the subject relative to a respiration cycle of the subject.

15. The system of claim 14, wherein the reference comprises one of a positioning assembly configured to support the imaged subject or an imaging system, and wherein the tracking system includes at least a first sensor positioned on the imaged subject, at least a second sensor located at a reference relative to a position of the subject, the second sensor configured to detect a position data of the first sensor relative to the second sensor.

16. The system of claim 14, wherein the gate signal comprises an ON state and an OFF state.

17. The system of claim 16, wherein the system compares the position of the first sensor relative to a threshold, and wherein the gate signal is in the ON state when the position of the first sensor is within the threshold, and wherein the gate signal is in the OFF state when the position of the first sensor is detected not within the threshold relative to the second sensor.

18. The system of claim 16, wherein the radiation source is configured to transmit radiation in response to detecting the ON state in the gate signal.

19. The system of claim 16, wherein the radiation source is configured not to transmit radiation in response to detecting the OFF state in the gate signal.

20. A system operable to navigate instruments relative to an image data of an imaged subject, the system comprising:
a respiratory cycle measurement device coupled to receive a position data of a first sensor attached to a patient in relation to a reference, wherein the respiratory cycle measurement device translates the position data relative to time into a respiratory signal representative of a respiratory cycle of the imaged subject; and
a controller operable to continuously reposition an image data relative to limits of a displayed image based on the position of the first sensor relative to the reference.
